(19) United States
(12) Patent Application Publication
(10) Pub. No.: US 2004/0001806 A1
(43) Pub. Date: Jan. 1, 2004

(54) Preparation of dendritic cells from spinal cord stem cells

(76) Inventor: Dirk Weickmann, Munchen (DE)

Correspondence Address: JENKINS & WILSON, PA, 3100 TOWER BLVD, SUITE 1400, DURHAM, NC, 27707, US

(21) Appl. No.: 10/292,152

(22) Filed: Nov. 12, 2002

Related U.S. Application Data
(63) Continuation of application No. PCT/EP01/05593, filed on May 16, 2001.

(30) Foreign Application Priority Data
May 17, 2000 (DE) 10024384.3

(51) Int. Cl. A61K 45/00; A61K 38/20; C12N 5/08

(52) U.S. Cl. 424/93.7; 435/368; 424/85.2

(57) ABSTRACT

The present invention describes a method for the preparation of dendritic cells wherein spinal cord stem cells grow in a nutrient medium in the presence of the growth factors GM-CSF and IL-4 and optionally in addition in the presence of the interleukin receptor sIL-4R until dendritic cells have formed. The thus prepared dendritic cells are preferably loaded with toxins, cytokins and/or cytokin receptors and are used in the treatment of tumor patients.

The invention relates to the preparation of dendritic cells from spinal cord stem cells by the addition of the growth factors GM-CSF and IL-4 as well as optionally in the presence of the interleukin receptor sIL-4R in vitro for therapeutic use in the case of tumor diseases, preferably cutaneous tumor diseases.

Prior Art

The term dendritic cells refers to cells of the immune system which have differentiated from stem cells and carrying dendrites which track and destroy genetically transformed cells. The destruction may for example occur by pumping of immune peptide toxins into the genetically transformed cell by means of the dendrites following docking to this cell. In this case the dendritic cell functions as a transport cell. The immune peptide toxin leads to the lysis of the transformed cell.

In the case of a weakened immune state the number of dendritic cells present within the body in a joint effort with the rest of the immune system is insufficient to effectively control transformed cells.

For the treatment of a locally manifested tumor, preferably a cutaneous tumor, a patient's own dendritic cells produced in vitro may be injected into the patient either into the tumor or around the tumor site. Preferably, these dendritic cells may additionally be loaded with cytokins and/or immunotoxins. For conventional dendritic cells prepared from blood stem cells, however, the loading capacity is limited.

At present dendritic cells are prepared from blood stem cells. Differentiation occurs in vitro upon addition of individual growth factors or growth factor mixtures, respectively.

A disadvantage of this method is the non-uniform differentiation resulting in an unequal basic cell volume. This renders loading with therapeutically effective substances from foreign species (animal and plant toxins) almost impossible.

It is known from Proc. Natl. Acad. Sci. USA, volume 93, pp. 2588-2592, 1996, to differentiate CD14⁺ blood monocytes to functional mature CD83⁺ dendritic cells. The specific combination suggested as a solution of the problem underlying the present invention is not anticipated or rendered obvious thereby. Particularly, this document does not anticipate the preparation of dendritic cells from spinal cord stem cells in combination with the growth factors to be used and the complementary addition of an interleukin receptor, respectively. In addition, the dendritic cells described in D1 do not show the good loading capacity with toxic substances as those prepared by the method according to the invention.

Therefore, it is an object of the present invention to provide an improved method useful in tumor therapy and/or as a supportive therapy e.g. in addition to conventional radiotherapy of tumor diseases and which avoids the disadvantages of the prior art mentioned above.

According to the invention this problem has been solved by a specific selection of the starting material (spinal cord stem cells) and by the use of a specific combination of growth factors (GM-CSF and IL-4) optionally in addition in combination with the interleukin receptor sIL-4R.

The dendritic cells prepared in this manner are characterized in particular by their uniformity and further may be loaded with therapeutically effective substances in an excellent manner both with respect to quantity and quality more efficiently than the dendritic cells prepared from blood stem cells of the prior art. They show these advantages also over the dendritic cells prepared from spinal cord stem cells without the use of the specific combination of growth factors described herein optionally also together with the interleukin receptor sIL-4R.

In the following the present invention will first be described generally and then in more detail with respect to an Example. The invention, however, is not limited to the specific embodiments described in the following specification and to the Example. Individual embodiments of the invention may be modified within the scope of the specification and the claims without parting from the spirit of the invention.

The method according to the present invention is based on human or animal spinal cord stem cells where it should be understood that human spinal cord stem cells are preferred. Preferably used are autologous stem cells which are reintroduced into the donor after differentiation to dendritic cells.

The spinal cord stem cells are isolated form spinal cord by methods known per se. Most widely used for this purpose are spinal cord puncture methods. The tissue obtained in this manner may be used directly for cell culture although generally, however, the spinal cord removed is treated to keep the cells viable for a prolonged period of time. For this purpose a treatment by shock-freezing has been particularly effective. The cells treated in this manner are then stored in liquid nitrogen. It should be understood that both the removal and the storage have to be performed in a way to ensure as much sterility as possible.

The spinal cord stem cells are introduced into conventional cell culture flasks into a medium suitable for growth. Typical media known to those skilled in the art for culturing spinal cord stem cells may be used. DMEM Ham's F-12 medium in combination with RPMI has been found to be particularly effective. To avoid contamination antibiotics are added, preferably mixtures of antibiotics. Antibiotics which may be used are for example penicillin and streptomycin. Further additives are amino acids, for example glutamine, and fetal calf serum or substitutes therefor. The cells are cultured at a suitable temperature in an incubator wherein body temperature of about 37° C. is particularly preferably used.

The cells are cultured in the presence of a specific combination of growth factors, namely GM-CSF and IL-4, and optionally also of the interleukin receptor sIL-4R. It has been found according to the present invention that only by this combination of growth factors and optionally in addition of the interleukin receptor uniform dendritic cells are formed which can be loaded with active substances, for example with cytokins, cytokin receptors and cytotoxic agents in a particularly effective manner. The growth factors added and the optionally additionally added interleukin receptor may be derived from natural sources or may be recombinant in nature. The amounts of growth factors added may be determined and optimized by those skilled in the art by means of manual experimentation. Generally, the amount of the growth factors added is 800-1000 U/ml per each growth factor while a range of about 860-950 U/ml has been found particularly suitable. The optional supplementary addition of the interleukin receptor sIL-4R is performed in an amount of 500-750 U/ml, further preferred in an amount of 550-700 U/ml, also preferred in an amount of 500-600 U/ml.

In a particularly preferred embodiment of the present invention, GM-CSF, IL-4 and sIL-4R are used for the preparation of the dendritic cells from spinal cord stem cells.

It is possible to add the growth factors and the receptor directly at the beginning of the culture of spinal cord stem cells. In a preferred embodiment of the present invention, however, the dendritic cells are cultured until the bottom of the cell culture flask is completely overgrown with at least one, preferably with up to several cell layers. At this time point the mixture of the growth factors as well as optionally sIL-4R are added. It should be understood that the growth of the cells has to be monitored at regular intervals and that the medium has to be replaced. The required techniques for this purpose are known for a long time and are available to those skilled in the art.

After several hours, generally at least 24, the differentiation of the spinal cord stem cells into dendritic cells may be observed by microscopic monitoring. The differentiated dendritic cells which are mobile and in contrast to the spinal cord stem cells have no tight connection to the bottom of the cell culture flask are then collected and transferred into a new cell culture flask. The old cell culture flask is filled with new medium for the differentiation process to continue. As soon as new spinal cord stem cells are no longer formed or the formation in a sufficient amount no longer occurs the culture is discarded. Generally, the differentiated dendritic cells may be cultured in the new cell culture flask for at least three further weeks and are available for therapeutic purposes. It is also possible, however, not to use the dendritic cells prepared according to the invention directly in therapy but to store them first deep-frozen and contact them with the other therapeutically effective substances, i.e. for example with the toxins, only directly prior to their use for loading.

The dendritic cells obtained by the method according to the present invention have an advantageous uniformity and can be loaded with foreign substances in a particularly advantageous manner. These foreign substances include for example cytokins, toxins, and cytokin receptors.

Examples of the cytokins useful according to the present invention are the interferons IFN-gamma/INF-gamma and/or IFN-alpha/INF-alpha and/or the interleukins IL-1, IL-2, IL-3, IL-4, IL-6, IL-10, IL-14, IL-17 or a combination thereof.

Furthermore, it has been found that the spinal cord stem cells prepared according to the present invention are loaded preferably with cytokin receptors. Preferably, interleukin receptors are used. By so far unknown mechanisms the cytokin receptors, preferably the interleukin receptors, initiate the formation of interleukins so that loading with these receptors is particularly reasonable. Examples of cytokin receptors are the interleukin receptors, preferably the interleukin receptors sIL-1R, sIL-2R, sIL-3R, sIL-4R, sIL-6R, sIL-10R, sIL-14R, sIL-17R. Particularly preferred is to load the dendritic cells with the interleukin receptors sIL-4R and/or sIL-6R.

Loading of the dendritic cells for example with toxins is known per se. Loading also includes the incorporation of substances into the dendritic cell and not only the mere adhesion of the substances onto the cell. One possible type of loading is lipofection. Originally, this technique has been used in the transformation of cells with foreign DNA or RNA. An increase in the efficiency of loading may be achieved by binding antibodies, proteins, glycolipids etc. to the liposomes thus influencing interactions between liposomes and cell surfaces in a targeted manner. In this way toxins, preferably peptide toxins, cytokins or cytokin receptors can be recognized as “belonging to the cell” and can be introduced into the dendritic cell during “normal” metabolic functions. Other methods of loading are injection into the cell or incubation of the cell in a medium containing the substances. Reference is made in this respect to e.g. Herder (1995), Lexikon der Biochemie und Molekularbiologie, volume 2, Spektrum Akademischer Verlag, Heidelberg.

Another method of loading is laser microinjection. In this method dendritic cells present in a medium containing toxic substances are treated with a highly focused laser beam for a short time generating a hole in the cell membrane through which the surrounding medium may enter the cells. The technique underlying this method of micromanipulation of cells or cell aggregates is described for example in Schütze K. et al.; Nature Biotechnology, 16, 8: 737-742 (1998), Schütze K. et al.; J. Cell. Mol. Biol., 44, 5: 735-746 (1998), Böhm M. et al.; Americ. J. Pathology; 151, 1: 63-67, Pontén F. et al.: Mutation Research Genomics, 382: 45-55 (1997), Bernsen M. et al.; Lab. Invest. 78: 1267-1273 (1998) or Fink L. et al.; Nat. Medicine. 4, 11: 1329-1333 (1998).

Another method for loading the dendritic cells is as follows:

A medium solution saturated with toxin (about 2 mg of the toxin fraction per 5 ml of cell culture medium) is added regularly to the previously differentiated cell cultures of the uniform dendritic cells prepared from spinal cord stem cells according to the present invention. If this is repeated every 2 days with medium exchange a toxin acceptance of the dendritic cells occurs which is accompanied by an incorporation of the toxins by the cells. If these dendritic cells are contacted with the transformed cells to the controlled a quickly initiated effect on the tumor cells can be observed brought about by the dendritic cells loaded with foreign substances. This method results in a very high loading capacity of the dendritic cells.

In a preferred embodiment of the invention there are used as the toxic substances those having a cytotoxic effect, necrotic effect and/or apoptotic effect. Particularly preferred substances used for this purpose are derived from the poisons of scolopenders, snakes, scorpions, and spiders. Dendritic cells loaded in this manner which have been prepared by the method according to the invention are especially useful for the therapy of cutaneous carcinoma, for example of melanomas. Preferably these are peptide toxins of the following animals: scolopender species Scolopendra morsitans, S. gigantea and Hemiscolopendra sp., of the snake species Bitis arietans (puff adder), Bitis gabonica (Gaboon viper), Bitis nasicornis (rhinoceros viper), and of the smaller Bitis species B. atropos, B. caudalis, B. peringueyi, and of the spitting cobra species Naja melanoleuca and Naja pallida as well as of the araneomorphic violin spider species Loxosceles laeta, L. spiniceps, L. bergeri, L. parami, L. rufescens, L. reclusa, L. deserta, the vibrating spider species Pholcus phalangioides, Pholcus opilionides, Pholcus spp. (RSA), Pholcus spp. (Cuba), Sicarius albospinosus, S. hahni, S. oweni, S. testaceus, S. spp (South America) as well as of the fat tail scorpion species Parabuthus transvallicus, P. granulosus, P. villosus. It is not required for this purpose that the toxins are present in pure form. It is also possible to fractionate the poisons, for example by column chromatographic methods, and to load the dendritic cells with the components of those fractions showing e.g. a cytotoxic effect.

In the following the present invention will be described in more detail with respect to an Example:

Method of Preparation

Preferably the human spinal cord stem cells are deep-frozen in Eppendorf sample vials (E cups) having a capacity of 1 ml. Preferably this is a saturated stem cell solution (saline solution or plasma, 50 microliters at maximum).

For further processing, preferably 750 microliters of medium (37 degrees Celsius) consisting of 500 ml DMEM Ham's F-12, 50 ml RPMI 1260, 10 ml penicillin/streptomycin, 5 ml glutamine, and 50 ml FCS are added.

The suspension diluted in this way is then agitated for 10-15 seconds on a vortex, lowest setting, without formation of foam to obtain homogenous mixing.

Directly afterwards the suspension is introduced into a 75 cm² cell culture flask and topped with 20 ml of the same medium (37 degrees Celsius).

Two to three times careful manual swinging to achieve an optimal distribution of the spinal cord stem cells present in the suspension in the cell culture flask.

This cell culture flask is then placed in an incubator set to 37 degrees Celsius.

It is preferable to leave the cell culture prepared for five days at a uniform temperature (37 degrees Celsius) in the incubator.

This enables adhesion of the spinal cord stem cells to the bottom of the flask.

The first visual monitoring by an inverted microscope as to whether the spinal cord stem cells have adhered to the bottom of the flask is preferably carried out five days after placing the cell culture flask in the incubator.

It is preferable to carry out the first medium exchange under sterile conditions five days after preparation of the cell culture as follows:

The medium flask is held in a way that as much as possible of the medium contained therein is removed without pouring out the already adhered cells at the same time. Optionally present residual medium is removed by gentle tapping of the bottleneck onto a pad.

The medium collected in this manner is discarded.

Subsequently, 20 ml of new medium are carefully introduced into the cell culture flask without releasing the adhered cells from the bottom of the cell culture flask.

The thus described medium exchange is to be carried out after every 3 or 4 days, respectively, until several cell layers have grown on top of each other and the first cells start to detach into the supernatant medium.

Growth of the cell culture is monitored by an inverted microscope prior to each medium exchange.

If the flask is overgrown with several cell layers at the bottom of the cell culture flask all the supernatant of about 20 ml is subcultured in equal amounts in 4 small 25 cm² flasks and is afterwards topped with medium warmed to 37 degrees Celsius.

These subcultures are left for five days at 37 degrees Celsius in the incubator without any further treatment.

Starting on the fifth day the medium exchange described above is carried out every three or four days, respectively, in each case with 5 ml medium under permanent microscopic control.

If the bottom of the cell culture flask is completely overgrown with cell layers, addition of the growth factors is carried out in such an amount that a concentration of 900 U/ml each GM-CSF and IL-4 (obtained e.g. from Biochrom company) is achieved. A temperature of 37 degrees Celsius is preferred during addition. The addition of the growth factor mixture as well as of 700 U/ml sIL-4R is performed using a sterile 2 ml pipette.

The cell culture flask is carefully swung manually without detaching the cells from the bottom.

For at least 24 hours the cell culture flask is stored at 37 degrees Celsius in an incubator.

Not before the elapse of 24 hours a first visual control is performed using an inverted microscope whether dendritic cells have already differentiated which are then transferred daily under highly sterile conditions using a glass Pasteur pipette with an as low amount of medium as possible into a new flask (25 cm²) containing medium (2 ml) already prewarmed to 37 degrees Celsius until no new dendritic cells develop within the original flask.

The medium exchange for the dendritic cells collected in the new cell culture flask is preferably carried out in a way that the old medium is aspirated with a Pasteur pipette under highly sterile conditions until only 1 ml is left without aspirating dendritic cells at the same time and is then filled with 4 ml of new medium prewarmed to 37 degrees Celsius.

This medium exchange is then performed every three or four days, respectively, in the manner described.

By the thus obtained dendritic cells having a viability of at least three weeks a sufficient number for therapeutic purposes is provided.

Loading of Dendritic Cells with Peptide Toxins

3-5 ml of a culture of dendritic cells (2.5 to 15 millions of dendritic cells/5 ml) prepared according to the method described above in the present invention were contacted with 0.5 to 2.5 ml of a cutaneaous carcinoma cell solution (supernatant of a freshly tapped pure human cutaneous carcinoma cell culture flask containing about 250,000 cutaneous carcinoma cells/ml cell medium, medium: 500 ml DMEM Ham's F-12, 10 ml penicillin/streptomycin, 5 ml glutamine and 50 ml FBS. Afterwards 1 to 2 ml of a solution containing about 150 μg/ml peptide toxin of fraction 3 derived from Loxosceles spiniceps were added. Incubation was carried out for 24 hours at room temperature. Then, using a previously autoclaved Pasteur pipette, about 4 ml of medium supernatant (if possible without dendritic cells) were removed under a sterile workbench and discarded. Subsequently another addition of 1 to 2 ml of the peptide toxin Lox.tox. 3 (same concentration as in the first addition) was performed resulting in a final concentration of peptide toxin of about 100 μg/ml. The dendritic cells loaded in this way are further incubated at 37° C. in an incubator until they are used.

The thus prepared dendritic cells, particularly those loaded for example with substances having a skin necrotic, cytotoxic and/or apoptotic effect are administered to the patient, preferably to the patient from whom the spinal cord stem cells were obtained, in the form of a pharmaceutical preparation. Administration of the dendritic cells loaded with foreign substances may be carried out in a manner known per se, for example by injection or infusion. It is also possible to implant the cells into the tumor tissue in the form of a depository so that the dendritic cells loaded with active agents are continuously delivered to the surrounding tumor tissue.

1. A method for the preparation of dendritic cells in vitro
characterized in that spinal cord stem cells are grown in a nutrient medium in the presence of the growth factors GM-CSF and IL-4 and optionally in the presence of the interleukin receptor sIL-4R until dendritic cells have formed.

2. A method according to claim 1
characterized in that human spinal cord stem cells are used.

3. A method according to claim 1 or 2
characterized in that the addition of the growth factors and of the interleukin receptor is carried out after the cell culture flask is completely overgrown with the spinal cord stem cells.

4. A method according to one or more of the preceding claims
characterized in that the growth factors each are added in an amount of 800-1000 U/ml.

5. A method according to claim 4
characterized in that the growth factors each are added in an amount of 860-950 U/ml.

6. A method according to one or more of the preceding claims
characterized in that the forming dendritic cells are removed from the cell culture flask in periodic intervals.

7. A method according to one or more of the preceding claims
characterized in that as the cell culture medium DMEM Ham's F-12, RPMI 1260 is used, optionally supplemented with antibiotics, glutamine and FCS.

8. A method according to one or more of the preceding claims
characterized in that the interleukin receptor is added in an amount of 500-750 U/ml.

9. A method according to one or more of the preceding claims
characterized in that sIL-4R is added in an amount of 550-700 U/ml.

10. Dendritic cells prepared from spinal cord stem cells using the growth factor combination GM-CSF and IL-4, optionally additionally in combination with the interleukin receptor sIL-4R.

11. Dendritic cells according to claim 10
characterized in that they are loaded with cytokins and/or cytokin receptors and/or toxins.

12. Dendritic cells according to claim 11
characterized in that as said toxins there are used peptide toxins having a cytotoxic, necrotic and/or apoptotic effect from the poison of scorpions of the genus Parabuthus, scolopenders of the genus Scolopendra, Hemiscolopendra, of snakes of the genus Bitis, Naja, and/or spiders of the genus Loxosceles, Lycbsa, Sicarius and/or Pholcus; and as said cytokins there are used the interferons IFN-gamma/INF-gamma and/or IFN-alpha/INF-alpha and/or the interleukins IL-1, IL-2, IL-3, IL-4, IL6, IL-10, IL-14, IL-17 or a combination thereof; and
as said cytokin receptors there are used the interleukin receptors sIL-1R, sIL-2R, sIL-3R, sIL4R, sIL-6R, sIL-10R, sIL-14R, sIL-17R or a combination thereof.

13. The use of dendritic cells according to one or more of the preceding claims for the treatment of tumors.

14. The use according to claim 13 for the treatment of cutaneous tumors.
